(19) United States
(12) Patent Application Publication
(10) Pub. No.: US 2021/0282646 A1
(43) Pub. Date: Sep. 16, 2021

(54) MEDICAL DATA MEASUREMENT SYSTEM

(71) Applicant: TAIDOC TECHNOLOGY CORPORATION, New Taipei City (TW)

(72) Inventors: Chao-Wang Chen, New Taipei City (TW); Li-Ping Yang, New Taipei City (TW); Yeong-Dar Chen, New Taipei City (TW); Yi-Hsin Huang, New Taipei City (TW)

(21) Appl. No.: 17/200,491

(22) Filed: Mar. 12, 2021

(30) Foreign Application Priority Data
Mar. 16, 2020 (TW) 109108656

(51) Int. Cl. A61B 5/01; A61B 5/00; A61B 5/1171; A61B 5/11; G16H 10/60

(52) U.S. Cl. CPC A61B 5/01; A61B 5/7264; A61B 5/6815; A61B 5/1176; G16H 10/60; A61B 5/742; A61B 5/7405; A61B 5/7455; A61B 5/1112; A61B 5/746

(57) ABSTRACT

The invention relates to a medical data measurement system. The medical data measurement system comprises a sensing module and an intelligent module. The sensing module is used to monitor a change of a data. The intelligent module receives the data of the sensing module, and comprises an identification module and a processing module. The identification module is used to identify and determine to drive the processing module to obtain a physiological value from the data. The sensing module can be an automatic forehead temperature measurement. Through the above, automatically measuring, tracking, and locating a measurement of a medical data can be achieved.

### BACKGROUND OF THE INVENTION

### 1. Field of the Invention

The present invention relates to a medical data measurement system, especially a system comprising a sensing module and an intelligent module. More particularly, the present invention relates to a system that could convert real-time image into a distance.

### 2. Description of Related Art

As the prevalence of COVID-19, the demand for body temperature measurement in major hospitals, customs, government agencies, schools, office buildings, and public places has increased. Nowadays, measuring body temperature at every entrance has become a mandatory routine. Traditionally, body temperature measurement requires using forehead or ear thermometers to measure body temperature manually. This is not only time-consuming, but the person who assists the measurement needs to contact hundreds or even thousands of people a day causing a potential concern for contact infection. In addition, one solution is using an infrared thermal imager, although it can reduce the risk of contact infection and can quickly measure multiple people simultaneously, during a large amount of people, it is easy to lose focus and difficult to accurately grasp the temperature for every individual. Furthermore, the infrared thermal imager is more expensive than ear and forehead thermometers, thus it is less affordable for the masses.

Therefore, how to avoid excessive contact infection for the measurement personnel, and can accurately and quickly determine the body temperature, meanwhile making sure the public can measure the temperature in a comfortable, fast, and accurate way, is the direction for the relevant industry to improve.

### SUMMARY OF THE INVENTION

In order to solve the above noted conventional problems, one aspect of the present invention is to provide a medical data measurement system, which comprises:
a sensing module, configured to monitor a data change, and
an intelligent module configured to receive the data of the sensing module and comprises an identification module and a processing module. Preferably, the identification module captures a real-time image of a subject and converts it into an identification value, and the identification value determines if a physiological value is obtained by the processing module.

In a preferred embodiment of the present invention, the medical data measurement system further comprises a stand, and the sensing module and the intelligent module are preferably configured on the stand.

In a preferred embodiment of the invention, the sensing module is a forehead temperature measuring device or an ear temperature measuring device, and the physiological value is a forehead temperature or an ear temperature. Furthermore, the physiological value may comprise a predetermine compensating data, which can compensate for a temperature error by comparing the forehead temperature or the ear temperature with a specific temperature. Preferably, the sensing module uses a blackbody furnace to compensate the temperature error. In another preferred embodiment of the present invention, the sensing module further comprises a first analytic unit to analyze the facial recognition results. Preferably, the facial recognition detects the relative distance between eyes, eyebrows, ears, nose and mouth or other facial features to determine the result.

In a preferred embodiment of the present invention, the processing module may further comprises a second analytic unit to analyze the physiological value, and categorize the physiological value into a normal interval or an abnormal interval. The second analytic unit may further comprises a warning unit, and the waring unit is activated when the physiological value falls within the abnormal interval. The warning unit may comprise an audio unit, a visual unit, a vibration unit or any combination of the above; and a storage unit to store the physiological value.

Preferably, the data from the sensing module comprises a measurement time. Preferably, the storage unit is a personal intelligent device, a terminal system or an online storage.

In a preferred embodiment of the present invention, the medical data measurement system further comprises a positioning module configured to locate a geographic location of the physiological value. Preferably, the positioning module combining with the processing module estimates a human flow in a geographic location and a distribution of the physiological values. More preferably, the positioning module further connects to a back-end system to track movements of the measurement subject. The back-end system may be a healthcare/hospital information system (HIS), a nursing information system (NIS), a laboratory information system (LIS), an electronic medical record (EMR), a point-of-care (POC) system, or a social media software.

Another aspect of the invention provides a medical data measurement system, comprises:
an automatic forehead temperature measuring device; and
an intelligent module for receiving data from the automatic forehead temperature measuring device, comprising:
   an identification module; and
   a processing module.

Preferably, the identification module is configured to identify the data and determine to drive the processing module accordingly. Preferably, the identification module further comprises a real-time image acquiring from a measurement subject.

In a preferred embodiment of the present invention, the automatic forehead temperature measuring device is configured to continuously detect a plurality of data, and the identification module is configured to identify the plurality of data, when one of the plurality of data exceeds a preset threshold, the processing module is driven to obtain a forehead temperature value from the data.

In a preferred embodiment of the present invention, the processing module further comprises a second analytic unit configured to analyze the forehead temperature value and categorize the forehead temperature value into a normal interval or an abnormal interval. The second analytic unit may further comprises a warning unit, and the warning unit is activated when the forehead temperature value falls within the abnormal interval. The warning unit may further comprise an audio unit; a visual unit; a vibration unit, or any combination of the above; and a storage unit to store the forehead temperature value. Preferably, the storage unit is a personal intelligent device, a terminal system or an online storage mode.

In a preferred embodiment of the present invention, medical data measurement system according to the above may further comprise a positioning module for locating a geographic location of the forehead temperature value. Preferably, the positioning module combining with the processing module estimates a human flow in the geographic location and a distribution of the forehead temperature values. More preferably, the positioning module further connects to a back-end system to track movements of the measurement subject, and the back-end system may be a healthcare/hospital information system (HIS), a nursing information system (NIS), a laboratory information system (LIS), an electronic medical record (EMR), a point-of-care (POC) system, or a social media software.

In yet a preferred embodiment of the present invention, a method for measuring medical data implemented by a medical data measurement system is disclosed, the system comprises a sensing module, an identification module and a processing module, and the method comprising the steps of:

sensing a plurality of data by the sensing module;
obtaining a real-time image of a measurement subject by the identification module;
converting the real-time image to a measurement distance;
determining the measurement distance exceeds a preset threshold; and
driving the processing module to obtain a physiological value from the plurality of data. Preferably, determining whether the measurement distance exceeds the preset threshold is to determine if the measurement distance is less than 20 cm. More preferably, determining whether the measurement distance exceeds the preset threshold is to determine if the measurement distance is less than 7 cm. Furthermore, sensing a plurality of data is sensing a plurality of temperature values, and the processing module is driven to obtain a forehead temperature value by the plurality of temperature values.

Another aspect of the invention provides a method for measuring medical data implemented by an automatic forehead temperature measurement system which comprises an identification module and a processing module, and the method comprises the steps of:
sensing a plurality of temperature values by the automatic forehead temperature measurement device;
receiving the plurality of temperature values;
determining if the plurality of temperature values exceeds a preset threshold; and
driving the processing module to obtain a forehead temperature value from the plurality of temperature values.
   Preferably, determining if the plurality of temperature values exceeds a preset threshold is to determine if the plurality of temperature values exceeds 35 degrees Celsius. Preferably, the method in accordance with the present invention can further comprises a step of: obtaining a real-time image of a measurement subject by the identification module. More preferably, the present invention further comprises a step of: displaying the real-time image and the forehead temperature. Furthermore, the method in accordance with the present invention further comprises a step of: storing the real-time image and the forehead temperature.

Another aspect of the invention provides a method for measuring medical data implemented by a medical data measurement system comprising a sensing module, an identification module and a processing module, and the method comprising the steps of:
sensing a plurality of values by the sensing module;
obtaining an identification value by the identification module;
determining if the identification value exceeds a preset threshold; and
driving the processing module to obtain a physiological value from the plurality of values. Preferably, the identification value in the medical data measurement system determines to drives the processing module to obtain a physiological value. More preferably, the physiological value is a forehead temperature.

In a preferred embodiment of the present invention, the identification module in the medical data measurement system is configured to obtain a real-time image of a measurement subject and convert it to an identification value, which is a distance. Based on the identification of the distance, the processing module is determined to obtain a physiological value from the data. Preferably, the physiological value is a forehead temperature value.

Based on the above, the medical data measurement system of the present invention uses the identification module to record an image, a time, and a body temperature and combined with automatic forehead temperature measurement, to achieve the purpose of traceability and greatly reduce excessive contact to infection sources.

In order to make the above-mentioned features of the present invention more understandable, embodiments of the invention are described in details below in conjunction with drawings.

### BRIEF DESCRIPTION OF THE DRAWINGS

Many aspects of the present disclosure can be better understood with reference to the following drawings. The components in the drawings are not necessarily to scale. Instead, emphasis is placed on illustrating the principles of the present disclosure.

FIG. 1 is a block diagram of a preferred embodiment of a medical data measurement system of the present invention.

FIG. 2 is a schematic view of a preferred embodiment of the medical data measurement system of the present invention.

FIG. 3A is a standby state diagram of a tablet of the medical data measurement system of the present invention.

FIG. 3B is a normal state diagram of the tablet of the medical data measurement system of the present invention.

FIG. 3C is a warning state diagram of the tablet of the medical data measurement system of the present invention.

FIG. 4 is a flowchart of a preferred embodiment of the medical data measurement system of the present invention.

### DESCRIPTION OF THE EMBODIMENTS

For the purposes of promoting an understanding of the principles of the invention, reference will now be made to the embodiment illustrated in the drawings, and specific language will be used to describe that embodiment. It will nevertheless be understood that no limitation of the scope of the invention is intended. Alterations and modifications in the illustrated device, and further applications of the principles of the invention as illustrated therein, as would normally occur to one skilled in the art to which the invention relates are contemplated, are desired to be protected. Such alternative embodiments require certain adaptations to the embodiments discussed herein that would be obvious to those skilled in the art.

As mentioned above, the person who assists the measurement needs to contact hundreds, even thousands of people a day. The risk of contact infection is very high, which may become a concern in epidemic prevention. In addition, measuring with an infrared thermal imager, although it can reduce the risk of contact infection and quickly measure multiple people simultaneously, is easy to lose focus due to a large number of people, and it is difficult to accurately grasp the temperature for every individual. Furthermore, the infrared thermal imager is more expensive than ear and forehead thermometers, thus it is less affordable for the masses.

On the contrary, an embodiment of the present invention comprises a sensing module to monitor a change of a data and an intelligent module to receive data from the sensing module and comprising an identification module to obtain an identification value. The identification module can obtain a real-time image of a measurement subject and convert it into an identification value. Alternatively, the identification module generates an identification value through the data and drives a processing module to obtain a physiology value by the identification value to achieve fast and accurate measurement. Hereinafter, embodiments of the present invention will be explained in detail with drawings. The drawings illustrate exemplary embodiments of the present invention, in which the same numerals indicate the same or similar elements.

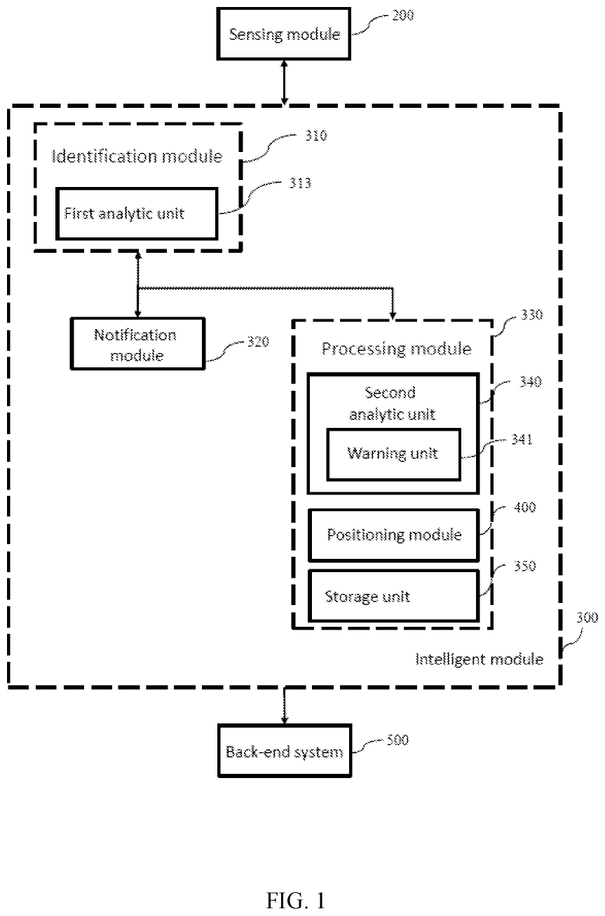

Please refer to FIG. 1, which is a block diagram of a preferred embodiment of a medical data measurement system 1 of the present invention. In the embodiment, a medical data measurement system 1 in accordance with the present invention comprises a sensing module 200 and an intelligent module 300. The medical data can be body temperature, heart rate, respiration rate, blood oxygen, etc., but the scope of the present invention is not limited thereto. The sensing module 200 monitors a change of a data, which can be obtaining a plurality of values or only a single value. The intelligent module 300 receives the data monitored by the sensing module 200 and determines whether the data should be stored.

Preferably, the sensing module 200 in accordance with the present invention can monitor a data change to obtain a physiological value. Alternatively, the sensing module 200 can automatically and continuously monitor a change of the data. Alternatively, the sensing module 200 can sense a data. More specifically, the sensing module 200 can obtain a plurality of data by automatically and continuously monitoring the data. Furthermore, a physiological value can be obtained from the plurality of data. Preferably, the sensing module 200 can be a temperature measuring device. The sensing module 200 is used to monitor a temperature change to obtain a single temperature value or a plurality of temperature values, more preferably, the sensing module 200 is a forehead temperature measurement device or an ear temperature measurement device. More specifically, the obtained physiological value is a forehead temperature value or an ear temperature value. However, the scope of the invention is not limited thereto. More specifically, the intelligent module 300 can be installed with a first initial algorithm, which enables the sensing module 200 to further identify the ambient temperature and someone passing in front of the sensing module 200 without performing a measurement or the calculation of temperature differences. For example, the disturbance of the ambient temperature can be set as a background value, and if someone passes in front of the sensing module 200, the raise of temporally temperature disturbance is set not to be measured. The actual measurement condition, which is staying in front of the sensing module 200 for a relatively long time, is set to perform a measurement. Furthermore, after the sensing module 200 performs the measurement, the physiological value will be stored. For example, if the relatively long time period is set to be 3 seconds, the temperature measured by the sensing module 200 will have to rise from the original ambient temperature to the user's body temperature and maintain said temperature for 3 seconds until the user moves away from the sensing module 200, and the temperature measured by the sensing module 200 drops back to the ambient temperature, which then completes a full measurement cycle. More specifically, the measurement value can only be regarded as a valid physiological value and be stored after the sensing module 200 measures a temperature exceeding a predetermined temperature threshold for an adjustable time period. Furthermore, the sensing module 200 can also train and refine its algorithm based on the data gathered from the first initial algorithm. For example, when a user wants to perform a measurement, but the sensing module 200 does not respond automatically, the user can manually press the measurement button to force the sensing module 200 to perform a measurement. At the same time, the intelligence module 300 can be trained and refined. As a result, when the same situation occurs next time, the sensing module 200 will automatically perform a measurement.

The intelligent module 300 in accordance with the present invention, preferably, can further comprise an identification module 310, a processing module 330 or a notification module 320. The identification module 310 can be used to obtain a real-time image (not shown in the figure) of a measurement subject to do a face recognition analysis. This is only an embodiment, and the invention is not limited thereto. More specifically, the face recognition analysis detects facial features or a relative distance of facial features for recognition. Preferably, the facial features are comprising of face size, cheekbones, temples, philtrum, corners of the mouth, jawline . . . as parameters for the face recognition analysis. More specifically, the intelligent module 300 comprises a second initial algorithm, which enables the identification module 310 to distinguish whether the face appears in the front is a human face based on the facial features. If no facial features are detected, no measurement will be performed. If the identification module 310 finds the aforementioned facial features, it can proceed to the next step of interpretation. The identification module 310 may further comprise a first analytic unit 313 for obtaining an identification value according to the face recognition analysis. Preferably, the identification value may be a measurement distance, which may be less than 20 cm, more specifically, the measurement distance is less than 7 cm, and more preferably, the distance is 3 to 5 cm. Take the face size as an example. Regardless of the size and race of human beings, the size of the head circumference is the smallest part of the appearance of each person. The width of the face of men and women without ears is between 12.5-15.9 cm, and the median value is between 13.3-14.5 cm. Take 13.9 cm as a characteristic parameter of each human face value for identification as an example. The first analytic device 313 can use a calculation formula I of an optical system, and the calculation formula I can be (focal length d)/(sensor size t)=(photographed Body length T)/(distance D). Replace the sensor size with the number of pixels on the image, which becomes a calculation formula II. The calculation formula II is distance (m)=focal length (mm)×subject size (m)÷image size (pixels), and then enter the known focal length, subject size, and image size to obtain the measurement distance. The notification module 320 compares the measurement distance to a preset threshold and displays a notification accordingly. For example, when the preset threshold is 7 cm and the measurement distance is greater than 7 cm, the notification module 320 indicates that the measurement distance must be shortened in order to perform the measurement. If the measurement distance is 3 to 5 cm, the processing module 330 is determined to be driven. Furthermore, the identification module 310 can also train and refine the second initial algorithm based on the conditions during the identification period of the second initial algorithm. For example, when a user enters into the measurable distance and is ready for a measurement, but the identification module 310 does not respond automatically, the user can manually press the measurement button to force the identification module 200 to perform a measurement. At the same time, the intelligent module 300 can be trained and refined. When the same situation occurs next time, the identification module 310 will automatically perform the identification and then perform the measurement.

The processing module 330 preferably further comprises a second analytic unit 340 and a storage unit 350. The second analytic unit 340 analyzes the physiological value and classifies the physiological value into a normal interval or an abnormal interval. Preferably, the data comprises a temperature value and a measurement time (not shown in the figure). Taking the temperature value as an example, the normal interval is 32 to 37.5 degrees Celsius, and the abnormal interval is above 37.6 degrees Celsius. Preferably, the second analytic unit 340 further comprises a warning unit 341. When the physiological value falls within the abnormal interval, the warning unit 341 will be activated. The warning unit 341 may comprise an audio unit, a visual unit, a vibration unit, or any combination of the above, but the invention is not limited thereto. The storage unit 350 is used to store the physiological value obtained by the sensing module 200. Preferably, the storage unit 350 is a smart personal device, a terminal system, or an online storage. On the other hand, the medical data measurement system 1 may further comprises a positioning module 400 for locating a geographic location of the physiological value. Preferably, the positioning module 400 may be a global positioning system (GPS) or a location-based services (LBS), and the positioning module 400 is used in conjunction with the processing module 330 to estimate the geographical location of the human flow and the physiological value distribution. Preferably, the positioning module 400 can further connect to a back-end system 500 to track the location status of the measurement subject. For example, the back-end system 500 is a healthcare/hospital Information system (HIS), a nursing information system (NIS), a laboratory Information system (LIS), an electronic medical record (EMR), a point-of-care (POC) system, or a social media software, but the scope of the invention is not limited thereto.

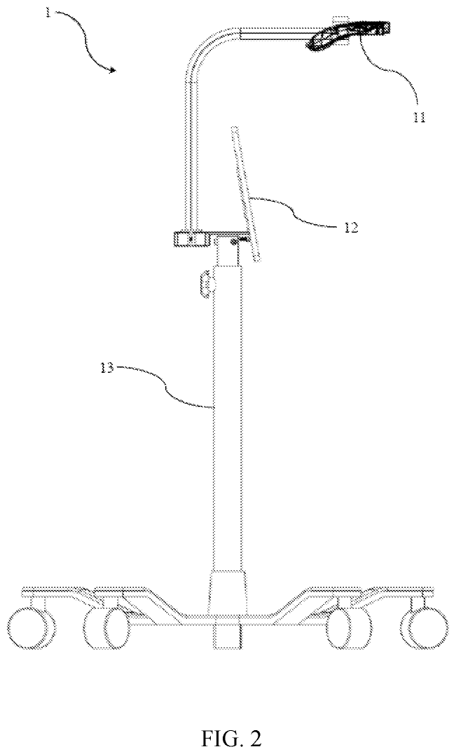

Please refer to FIG. 2, which is a schematic perspective view of a preferred embodiment of the medical data measurement system 1 according to the present invention. In this embodiment, the present invention provides a medical data measurement system 1 for monitoring a data and storing a physiological value of the data. Alternatively, it can also be used to locate a geographic location of a measurement subject. The medical data measurement system 1 comprises a tablet 12, a forehead thermometer 11, and a stand 13. Both the tablet 12 and the forehead thermometer 11 are set on the stand 13 and the configuration is not limited as long as the tablet 12 can receive the data from the forehead thermometer 11. Preferably, the forehead thermometer 11 is set on a top of the stand 13. More preferably, the forehead thermometer 11 is set on the stand 13 by a flexible arm with the tablet 12 set on an easily visible place and can be mounted on the stand 13 through a clip. Therefore, when a measurement subject approaches, the forehead thermometer 11 is leveled at an appropriate forehead height and the measurement subject can comfortably observe the tablet 12. The forehead thermometer 11 can automatically and continuously monitor temperature changes. The tablet 12 can be installed with an application program for receiving the temperature measured by the forehead thermometer. An identification module in the application program can determine whether to drive a processing module to obtain and store a temperature data. The application can access a camera function to further analyze based on a real-time image to know whether the measurement subject has entered the measurement range of the forehead thermometer 11. Preferably, the tablet 12 can be set in a place that is capable to obtain a real-time image. More specifically, the forehead thermometer 11 can have a wired or wireless transmission function and the monitored temperature can be transmitted to the tablet 12 by the wired or wireless transmission function. Preferably, the forehead thermometer 11 has a wireless Bluetooth function and the tablet 12 can receive the data monitored by the forehead thermometer 11 through the Bluetooth function. On the other hand, preferably, the stand 13 may be a movable stand; more preferably, a rotatable unit is attached at the bottom of the stand 13; specifically, the bottom of the stand 13 has wheels.

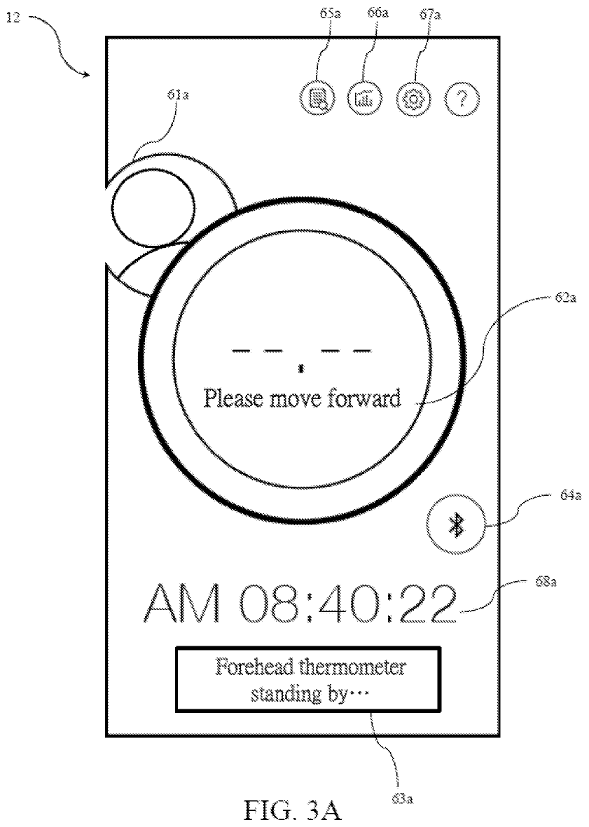

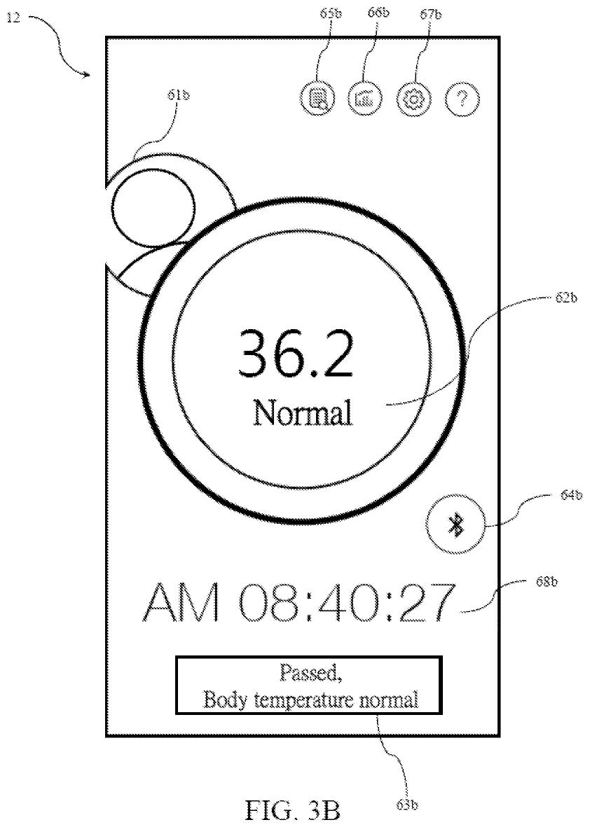

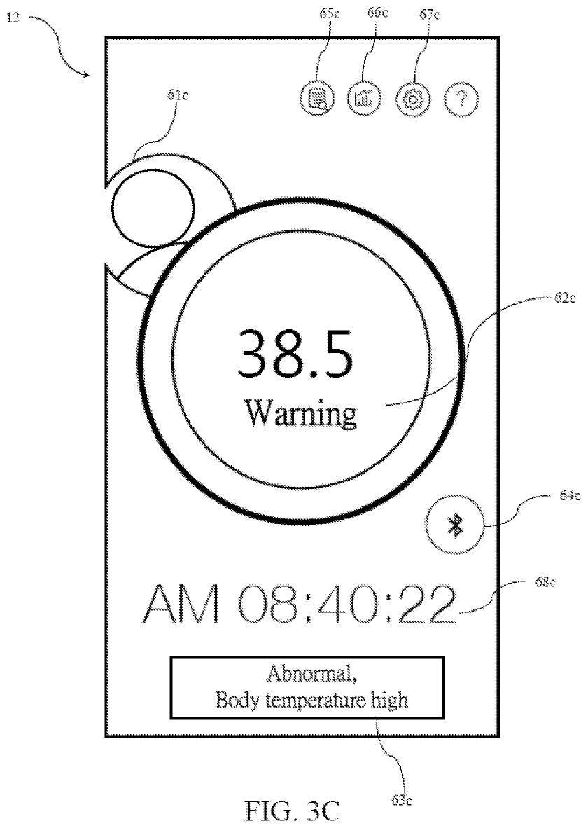

The FIG. 3A is a standby state diagram of the tablet of the medical data measurement system according to the present invention. FIG. 3B is a normal state diagram of the tablet of the medical data measurement system according to the present invention. The FIG. 3C is a warning state diagram of the tablet of the medical data measurement system of the present invention. Please refer to FIG. 3A to FIG. 3C in combination. In this embodiment of the present invention, the tablet 12 displays a real-time image 61a, 61b, 61c, a temperature 62a, 62b, 62c, a status 63a, 63b, 63c, a Bluetooth function 64a, 64b, 64c, a recording function 65a, 65b, 65c, an analysis function 66a, 66b, 66c and a setting function 67a, 67b, 67c. Preferably, the temperature 62a, 62b, 62c further comprises a temperature value and a text string. For example, when the tablet 12 is in the standby state, the text string is displayed as “please move forward” and the temperature value is not displayed. The status 63a is displayed as “forehead thermometer standing by”. When the measurement subject is within the measurement range, the medical data measurement system 1 measures the forehead temperature of the measurement subject. If the temperature value falls in the normal interval, the text string displays normal, and the status 63b displays “passed, body temperature normal”. On the contrary, if the temperature value falls in the abnormal interval, the text string displays as warning, and the status 63c is displayed as “abnormal, body temperature high”. Preferably, the recording functions 65a, 65b, 65c are used to record the real-time images 61a, 61b, 61c, the temperature 62a, 62b, 62c, the measurement time 68a, 68b, 68c and the geographic location for traceability. Preferably, the analysis functions 66a, 66b, 66c can analyze the data recorded by the recording functions 65a, 65b, 65c. More specifically, the analysis functions 66a, 66b, 66c can classify, sort, and perform a further statistic and estimate the human flow of the geographic location and the distribution of physiological values to facilitate subsequent tracking. Preferably, the setting functions 67a, 67b, 67c can adjust the temperature range based on the risk to respond to various situations.

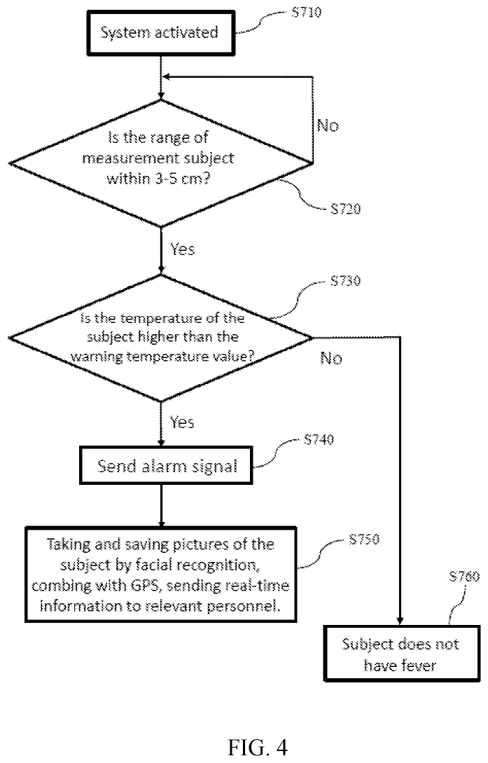

FIG. 4 is a flowchart of a preferred embodiment of a medical data measurement system according to the present invention. Please refer to FIG. 1 to FIG. 4 together. In this embodiment of the present invention, step S710, the forehead thermometer 11 is activated. In step S720, the identification module 310 analyzes the measurement distance, performs a face recognition analysis, and determines whether the measurement distance of the measurement subject falls within a desirable range. Preferably, the measurement distance is less than 20 cm, and more preferably, the measurement distance is less than 7 cm. In an embodiment of the present invention, the measurement distance is 3 to 5 cm. If the result is negative, the identification module 310 will indicate the notification module 320 to sense again, otherwise, if the result is positive, step S730 is executed. In step S730, the processing module 330 analyzes the temperature value of the measurement subject measured by the sensing module 200, and further determines whether the temperature value of the measurement subject is higher than the normal interval. Preferably, when the temperature value is higher than 37.6 degrees Celsius and the result is positive, the medical data measurement system 1 executes step S740 to send a warning signal. Step S750 continues following S740 by taking and saving pictures by the face recognition analysis. It can be further integrated with a global positioning system (GPS) positioning to upload the information of the suspected fever subject in real-time and provide it to relevant personnel for a follow-up. Conversely, if the determination in step S730 is negative, indicating that the temperature value is not higher than the normal interval, step S760 is executed to determine that the measurement subject does not have fever.

On the other hand, in another embodiment of the present invention, in step S720, the identification module 310 can identify whether the temperature measured by the forehead thermometer falls within a desirable range. If the temperature falls within the range then move on to step S730. Preferably, the range is over 34 degrees Celsius, more preferably, over 35 degrees Celsius.

Although the above embodiments have demonstrated possible forms of the invention for the medical data measurement system, people having ordinary skill in the art should know that the design of the medical data measurement system varies from manufacturers. Therefore, the present invention should not be limited to forms demonstrated above. In other words, as long as the medical data measurement system comprises a sensing module and an intelligent module, and the sensing module that can automatically measure medical data, preferably a temperature measurement, or the intelligent module can use image recognition to determine a distance, is already in line with the spirit of the present invention. Several examples are given below so that people having ordinary skill in the art can further understand the spirit of the present invention and how it can be implemented.

In the embodiment of FIG. 1 above, preferably, converting the real-time image to the identification value is only an optional embodiment. People having ordinary skill in the art can convert different information into the identification value for their needs. For example, the identification value can be a temperature value. The identification module 310 identifies whether the received temperature value exceeds a preset threshold value and if it exceeds the preset threshold value, the processing module 330 is determined to be driven. It could also notify the notification module 320 to display a notification. More specifically, when the temperature value exceeds a preset threshold, the notification module 320 displays “measuring” as a notification. During the test, the processing module 330 analyzes the physiological value with the help of the second analytic unit 340. Taking the embodiment of FIG. 2 as an example, the automatic measurement of the forehead temperature measurement device continuously performs a plurality of detections. When one of detection exceeds a preset threshold, preferably, the threshold can be set in advance to set its temperature range, the processing module is further determined to be driven to obtain a temperature measurement data.

In the embodiment of FIG. 1 above, preferably, the data of the sensing module 200 further comprises a predetermined compensative information of comparing the forehead temperature or the ear temperature with a specific temperature to compensate for a temperature error. More specifically, taking the forehead thermometer as an example, the predetermined compensation information is obtained by comparing the data measured by the forehead thermometer 11 with the specific temperature. Preferably, the specific temperature can be determined by a blackbody furnace. More preferably, the specific temperature is set to 35 degrees Celsius. Using the forehead thermometer 11 to measure the cavity inside the blackbody furnace, if the temperature measured by the forehead thermometer exceeds the temperature error, preferably ±0.2 degrees Celsius, the predetermined compensation information is transmitted to compensate for the error temperature. Preferably, the predetermined compensation information is preset in the intelligent module 300. However, it is only an optional embodiment and people having ordinary skill in the art can change the preset compensation information in the forehead thermometer 11 according to their needs.

To summarize, the present invention relates to a medical data measurement system that converts real-time images into to a measurement distance, and then drives the processing module based on the measurement distance or the determined temperature. It can automatically detect body temperature and reduce frequent contact between people, thus reducing the risk of disease transmission. In addition, the preferred embodiments of the present invention can also comprise the followings:

1. The medical data measurement system of the present invention can be set on a mobile stand with wheels, which is easy to be placed indoors or outdoors and can be moved freely. These make it easier to plan moving lines for the human flow.

2. The medical data measurement system of the present invention can have both automatic measurement and warning functions. One person can monitor up to 10 or more systems. As a result, the medical data measurement system offers an advantage of faster detection and saves more manpower and time.

3. The medical data measurement system of the present invention can only take 4 seconds/person from subject entering to leaving the measurement range. If only the measurement time is considered, it only takes 2 seconds/person. It can save time effectively and measure a large number of people fast.

Although the present invention has been disclosed as above with examples, it does not limit the present invention. Any person having ordinary skill in the art can make some changes and modifications without departing from the spirit and scope of the present invention. Therefore, the scope of the present invention shall be deemed as what the appended patent claims had disclosed.

What is claimed is:

1. A medical data measurement system, comprising:
a sensing module configured to monitor a data change; and
an intelligent module configured to receive the data from said sensing module, further comprising:
an identification module; and
a processing module, wherein said identification module captures a real-time image of a measurement subject and determines an identification value in accordance with the real-time image, and further determines said processing module in accordance with said identification value to obtain a physiological value from said data change.

2. The medical data measurement system of claim 1, further comprising a stand, wherein said sensing module and said intelligent module are configured to said stand.

3. The medical data measurement system of claim 1, wherein said sensing module is a forehead temperature measurement device or an ear temperature measurement device, and said physiological value is a forehead temperature or an ear temperature.

4. The medical data measurement system of claim 3, wherein said physiological value comprises a compensative information, wherein said compensative information is configured to compare said forehead temperature or said ear temperature with a particular temperature to compensate for a temperature error.

5. The medical data measurement system of claim 1, wherein said identification module further comprises a first analytic device, wherein said first analytic unit is configured to a facial recognition to obtain the identification value.

6. The medical data measurement system of claim 5, wherein said facial recognition is determined by the relative distance between eyes, eyebrows, ears, nose and mouth or other facial features.

7. The medical data measurement system of claim 1, wherein said processing module further comprising:
a second analytic unit configured to analyze said physiological value and categorize said physiological value into a normal interval or an abnormal interval, wherein said second analytic unit further comprising:
a warning unit, wherein said waring unit is activated when said physiological value falls within the abnormal interval, wherein said warning unit comprises an audio unit, a visual unit, a vibration unit or any combination of the above; and
a storage unit configured to store said physiological value.

8. The medical data measurement system of claim 7, wherein said storage unit is a personal intelligent device, a terminal system, or an online storage.

9. The medical data measurement system of claim 1, wherein said physiological value comprises a temperature value and a measurement time.

10. The medical data measurement system of claim 1 further comprising a positioning module configured to locate a geographic location of said physiological value.

11. The medical data measurement system of claim 10, wherein said positioning module combining with said processing module estimates a human flow in a geographic location and a distribution of said physiological values.

12. The medical data measurement system of claim 10, wherein said positioning module further connects to a back-end system to track movements of said measurement subject, wherein said back-end system is a healthcare/hospital information system (HIS), a nursing information system (NIS), a laboratory information system (LIS), an electronic medical record (EMR), a point-of-care (POC) system, or a social media software.

13. A medical data measurement system, comprising:
an automatic forehead temperature measuring device; and
an intelligent module for receiving a data from said automatic forehead temperature measuring device, comprising:
an identification module; and
a processing module, wherein said identification module is configured to identifying said data to determines said processing module is driven.

14. The medical data measurement system of claim 13, wherein said identification module further acquires a real-time image from a measurement subject.

15. The medical data measurement system of claim 13, wherein said automatic forehead temperature measuring device is configured to continuously detect a plurality of data, and said identification module is configured to identify said plurality of data, when one of said plurality of data exceeds a preset threshold, said processing module is driven to obtain a forehead temperature value from said data.

16. The medical data measurement system of claim 15, wherein said processing module further comprises:
a second analytic unit configured to analyze said forehead temperature value and categorize said forehead temperature value into a normal interval or an abnormal interval, wherein said second analytic unit further comprises:
a warning unit, wherein said warning unit is activated when said forehead temperature value falls within said abnormal interval, wherein said warning unit further comprises an audio unit, a visual unit, a vibration unit, or any combination of the above; and
a storage unit configured to store said forehead temperature value.

17. The medical data measurement system of claim 16, wherein said storage unit is a personal intelligent device, a terminal system or an online storage.

18. The medical data measurement system of claim 15 further comprising a positioning module for locating a geographic location of said forehead temperature value.

19. The medical data measurement system of claim 18, wherein said positioning module combining with the processing module estimates a human flow in said geographic location and a distribution of said forehead temperature values.

20. The medical data measurement system of claim 18, wherein said positioning module further connects to a back-end system to track movements of said measurement subject, wherein said back-end system is a healthcare/hospital information system (HIS), a nursing information system (NIS), a laboratory information system (LIS), an electronic medical record (EMR), a point-of-care (POC) system, or a social media software.
